(19) United States
(12) Patent Application Publication
(10) Pub. No.: US 2009/0133166 A1
(43) Pub. Date: May 21, 2009

(54) Methods for Improving Flowering Characteristics Using Methyl Dihydrojasmonate

(75) Inventors: Barbara Scheer, San Francisco, CA (US); Justin Scheer, San Francisco, CA (US)

Correspondence Address: PATENTBEST, 4600 ADELINE ST., #101, EMERYVILLE, CA, 94608, US

(73) Assignee: NEW BIOLOGY, INC., Burlingame, CA (US)

(21) Appl. No.: 12/272,060

(22) Filed: Nov. 17, 2008

Related U.S. Application Data
(60) Provisional application No. 60/988,960, filed on Nov. 19, 2007.

(51) Int. Cl. A01N 3/02; A01N 37/00; A01H 5/02

(52) U.S. Cl. 800/323; 504/313; 504/114

(57) ABSTRACT

Methods and formulations for improving the flowering characteristics of plants, such as rose plants, are disclosed. The formulations include methyl dihydrojasmonate:

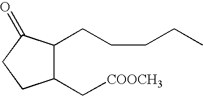

Methods and formulations according to embodiments of the invention may increase the duration that cut flowers last, increase the degree to which buds open to full flower, and increase the turgidity of stems. They may also prolong flower opening on intact flowering plants.

### CROSS-REFERENCE TO RELATED APPLICATIONS

This application claims priority to, and the benefit of, U.S. Provisional Patent Application No. 60/988,960, filed Nov. 19, 2007, the contents of which are incorporated by reference in their entirety.

### BACKGROUND OF THE INVENTION

1. Field of the Invention

Generally speaking, the invention relates to the field of plant biology, and more particularly, to methods for improving flowering characteristics using methyl dihydrojasmonate.

2. Description of Related Art

The jasmonates are a family of compounds related to jasmonic acid, 2-(3-oxo-2-(pent-2-enyl)cyclopentyl)acetic acid, the structure of which is shown below in Formula (1):

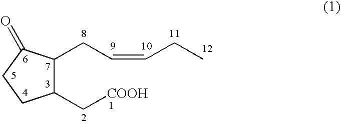

Jasmonates have been implicated in regulating a number of events in plant growth and development, as well as numerous types of plant responses to stressors. Osmotic stress or desiccation, touch, elicitation, wounding and pathogen and insect attack are all generally accompanied by increases in endogenous levels of jasmonates. Jasmonates are also widely used as flavoring and fragrance compounds because of their strong odor and taste characteristics.

Because of their apparent importance in plant life cycle events and stress responses, there have been studies of the relative bioactivity of various jasmonate compounds in single plant species (e.g., Miersch et al., Phytochemistry 50 (1999), pp. 353-361). There have also been studies of selected jasmonate compounds across multiple species (e.g., Gundlach and Zenk, Phytochemistry 47 (1998), pp. 527-537).

One jasmonate that has not been widely studied is the 9,10-dihydro methyl ester of jasmonic acid, commonly referred to as methyl dihydrojasmonate (MDHJ), the general structure of which is given below in Formula (2):

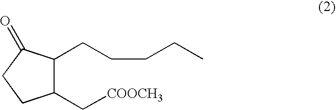

In general, the studies that have been done on MDHJ show that it is often less bioactive than other members of the jasmonate family, particularly methyl jasmonate (MJ), the methyl ester of jasmonic acid. Thus, there has not been a great focus on the properties and effects of MDHJ.

### SUMMARY OF THE INVENTION

One aspect of the invention relates to a method for improving flowering characteristics in a plant, comprising treating the plant with an effective amount of methyl dihydrojasmonate. In some embodiments, the method may further comprise harvesting one or more flowers from the plant after treating the plant with the effective amount of methyl dihydrojasmonate. The effective amount of methyl dihydrojasmonate may be in a form selected from the group consisting of emulsion, suspension, powder, hydrate, solution, granules, paste, aerosol, and volatile formulation.

Another aspect of the invention relates to a cut flower produced by a process including, prior to cutting the flower from a plant, treating the plant with an effective amount of methyl dihydrojasmonate.

Yet another aspect of the invention relates to a method for improving vase life of cut roses. The method comprises, prior to harvesting the roses from a rose plant, treating the rose plant with an effective amount of methyl dihydrojasmonate, and harvesting roses from the rose plant at a defined time after treating the plant.

Other aspects, features, and advantages of the invention will be set forth in the description that follows.

### BRIEF DESCRIPTION OF THE DRAWINGS

The invention will be described with respect to certain drawing figures, in which:

FIGS. 1 and 2 are sets of photographs illustrating the effects of treating roses with MDHJ prior to harvesting flowers; and

FIG. 3 is a set of photographs illustrating the effects of treating intact flowering plants with MDHJ.

### DETAILED DESCRIPTION

The present inventors have found that exogenous 9,10-dihydromethyl jasmonate (MDHJ), administered to a flowering plant in an effective amount, improves flowering characteristics. MDHJ may, for example, be applied to the plant prior to harvesting flowers. Flowering characteristics that may be improved in cut flowers include the duration that flowers last, the degree to which buds open into full flower, and the presence or absence of bent neck (bending of the peduncle below the bloom). These characteristics determine, at least in part, the “vase life” of a cut flower. In uncut flowering plants, MDHJ has also been found to prolong flower opening.

Plants to which MDHJ may be applied include, but are not limited to, angiosperms, monocots, dicots, roses, crop plants, ornamental plants, shrubs, trees, exotic plants, house plants, and native plants in cultivated or natural environments. MDHJ has been found to be particularly efficacious in roses.

The MDHJ may be applied alone or in a formulation comprising other elements, compounds, or substances. Some examples of other compounds that may be included in the formulation include wetting agents, adjuvants, emulsifiers, dispersants, spreaders, stickers, pastes, anchorage agents, fixatives, extenders, coating agents, buffering agents, plant nutrients, absorptive additives, and disintegrants. Those of skill in the art will recognize that a single ingredient may perform multiple functions, and may thus be classified or grouped in different ways. If the MDHJ is applied in the form of a foliar spray, it is generally desirable to include at least one exposure-increasing ingredient; i.e., at least one whose purpose is to increase the plant's exposure to the MDHJ or, more generally, to increase the influence of MDHJ on the plant. That exposure-increasing ingredient may be a wetting agent, a dispersant, a spreader, a sticker, an anchorage agent, a fixative, an extender, a coating agent, or an ingredient that acts by some other mechanism to increase plant exposure to MDHJ or to increase the influence of MDHJ on the plant. Exposure-increasing ingredients may or may not have discernible physiological effects on the plant when administered alone.

Particular examples of formulation ingredients include ionic, non-ionic, and zwitterionic surfactants, such as an octylphenoxypolyethoxyethanol-based surfactant like TRITON® X-100, TRITON® X-114, NP-40, SILWET, and sodium dodecyl sulfate; alcohols; organic solvents; synthetic or natural oils, such as castor oil, canola (rapeseed) oil, and soybean oil; soaps; and naturally derived adjuvants such as lecithin, saponin, and extracts from yucca, coconut, and pine. Each of these ingredients may be considered an exposure-increasing ingredient for purposes of this description.

In some embodiments, it may be beneficial to use ingredients that are high in compounds that play a role in the octadecanoic pathway. For example, canola oil is high in linoleic and linolenic acids, compounds that play a role in the octadecanoic pathway. Soaps of linoleic and linolenic acids may also be desirable formulation ingredients in some embodiments.

A formulation according to embodiments of the invention may also include fixative and extender compounds, in order to reduce volatility and evaporation of the active ingredient or ingredients, so as to increase exposure of the plant to the active ingredient. Exemplary fixatives include canola oil, castor oil, benzoyl benzoate, benzyl salicylate and synthetic musks, and sandalwood. Gums, waxes, and other carbohydrates, such as carnauba wax, carob gum, dextrins, dextrose, gellan gum, guar gum, paraffin wax, sorbitol, xanthan gum, polyvinylpyrrolidone, and glycerin, may also be used as fixatives.

Absorptive additives may also be included for extending the release and exposure time. Exemplary absorptive additives include, but are not limited to, silica gel; precipitated crystalline-free silica gel; amorphous, fumed, crystalline-free silica; amorphous, precipitated gel silica; silica hydrate; vitreous silica; silicic acid; and silicon dioxide.

Alone or in combination with other ingredients, the MDHJ may be delivered in the form of emulsions, suspensions, powders, hydrates, aqueous solutions, granules, pastes, aerosols, and volatile formulations. Any of these forms may be adapted for application to the plant's foliage, roots, stems, flowers, or any other portion of the plant that is capable of absorbing it. Particularly advantageous forms include foliar sprays, root solutions, and pellet-based root preparations. As a root solution or preparation, MDHJ may be formulated and applied to plants grown in soil, non-soil, artificial growing media, and/or hydroponic systems. In some embodiments, the MDHJ formulations may be combined with other active compounds that can be administered in the same fashion as the MDHJ formulation. Examples include fertilizers, seaweed, kelp, humic acid, and microbes. An MDHJ foliar spray may be combined with a foliar fertilizer, and a root solution may be combined with a fertilizer that is applied to the roots. Specific fertilizer and plant nutrient elements include, but are not limited to, nitrogen, potassium, phosphorus, calcium, magnesium, which may be compounded in any known manner so as to be absorbable by the plant. For example, plant nutrients may include monobasic potassium phosphate (KH₂PO₄) and magnesium sulfate (MgSO₄). MDHJ and MDHJ-containing preparations may be used in addition to other common growing and pre-harvest preparations, such as fertilizers, pesticides, and flower-life extenders.

As was described briefly above, the MDHJ would be applied in an “effective amount” to improve flowering characteristics. Effective amounts of MDHJ will vary from species to species and cultivar to cultivar, and will depend on the manner of application, the environmental conditions around the plant or plants, the form in which the MDHJ is administered, and the nature and type of additive compounds, if any, present in the formulation with the MDHJ. For example, if an MDHJ formulation is applied over a substantial portion of a plant's foliage, or is applied using a formulation that includes wetting agents, fixatives, and/or other additives intended to increase the level of exposure of the plant to the MDHJ, the formulation itself may contain a smaller amount or lower concentration of MDHJ than if an MDHJ formulation is applied over only a small portion of a plant's foliage, or without additives intended to increase the plant's exposure to the MDHJ. Similarly, if the MDHJ is administered in a form that tends to dwell on the plant's foliage, or in proximity to another part of the plant, then it may be administered in a lower concentration or amount.

As one example, an effective amount of MDHJ may comprise an aqueous solution with an MDHJ concentration in the range from about 0.15 mM to about 5 mM, inclusive. However, in some situations, and in some species, concentrations up to about 10 mM may be used. As those of skill in the art will realize, in general, MDHJ may be used in even higher concentrations for some applications, provided that the total dose of MDHJ that is absorbed by the plant is not phytotoxic. Similarly, lower concentrations may be adequate in some situations, for example, in an enclosed environment or greenhouse. As another example, the effective amount may comprise an aqueous solution with an MDHJ concentration in the range from about 1.5 mM to about 5 mM, inclusive.

One example of an aqueous MDHJ foliar spray formulation suitable for direct application to plants is given below in Table 1.

TABLE 1
Exemplary Aqueous Foliar Spray Formulation

| | | | % by | % by |
|---|---|---|---|---|
| Ingredient | g/L | ml/L | weight | volume |
| Water | 993.411 | 993.411 | 99.2931% | 99.341% |
| Methyl | 0.339 | 0.339 | 0.0339% | 0.034% |
| Dihydrojasmonate | | | | |
| (1.5 mM) | | | | |
| Canola Oil | 4.600 | 5.000 | 0.4598% | 0.500% |
| Triton X-100 | 1.325 | 1.250 | 0.1324% | 0.125% |
| KH₂PO4 - 4 mM | 0.544 | | 0.0544% | |
| MgSO4 - 0.8 mM | 0.197 | | 0.0197% | |
| Citric Acid - 0.347 mM | 0.067 | | 0.0067% | |
| Total | 1000.483 | 1000.000 | 1.000 | 1.000 |

In addition to liquid and aqueous preparations, MDHJ may be formulated for use in a slow-release application and provided in a granular- or pellet-based form, including fertilizer and/or pesticide formulations. Concentrations of MDHJ may be effective in weight/weight ratios to other ingredients in the range of about 0.008% to about 0.8%, and in some cases an effective ratio could be greater than 1.0% or less than 0.008%. Other inert or nutritive ingredients included in the pellets or granules can include binding agents and polymers such as polysaccharides and polyvinylpyrrolidone at 5-95%, a surfactant at 0.001-10%, and other absorptive ingredients such as acrylamide and acrylamide polymers.

Regardless of the concentration or amount of MDHJ in preparations intended for use, MDHJ formulations according to the present invention may be provided in the form of concentrates, so as to make shipping and distribution more efficient, and the task of preparing an appropriate suspension, solution, or other formulation for application may be left to the end user.

Formulations including MDHJ may be applied once or repeatedly, depending on the circumstances. For example, MDHJ formulations according to embodiments of the invention may be applied at regular intervals, such as every 10-14 days, every 30 days, or 1-2 times per month. In some embodiments, a single application of MDHJ prior to harvesting flowers may be used. For example, MDHJ may be sprayed on the flowering plants about 24 hours prior to flower harvesting.

One of the factors that may necessitate reapplication of an MDHJ formulation is the environmental conditions around the plant. For example, if the plants are field-grown or otherwise exposed to the elements, rain showers, excessive wind gusts, or other environmental factors shortly after an application may make a subsequent application desirable. Under some circumstances, a more dilute formulation or solution may be used if repeated applications are to be performed. Additionally, the effective amount of MDHJ for a plant that is grown outdoors may be different, e.g., greater, than the effective amount of MDHJ for a plant that is grown indoors or in a greenhouse. In some cases, an aqueous solution of MDHJ, with or without exposure-increasing ingredients, may be applied in vapor form within a greenhouse or other type of growing enclosure by allowing the vapor to enter the greenhouse without specifically targeting it to any one plant or group of plants.

The work of the present inventors has also shown that MDHJ can be used for other purposes, for example, to treat and prevent biotic attack in plants. U.S. patent application Ser. No. 12/235,654, filed Sep. 23, 2008, discloses the use of MDHJ to treat biotic attack, and is incorporated by reference herein in its entirety. If MDHJ is being applied to a plant for another purpose, the amount of MDHJ applied to the plant for that purpose may be sufficient to improve flowering characteristics. However, as will be set forth in the examples below in greater detail, in many embodiments, a single treatment with MDHJ may be sufficient to improve flowering characteristics. Generally speaking, MDHJ may be administered in addition to any other conventional additives or compounds typically used in cultivating plants, such as pesticides, fertilizers, and flower life extenders, to name a few.

### EXAMPLES

The following examples serve to illustrate the efficacy of MDHJ in improving flowering characteristics.

Unless otherwise noted, in the following examples, the MDHJ was obtained from Bedoukian Research, Inc. (Danbury, Conn., United States; product no. 398E). As supplied, the MDHJ solution was specified as having a minimum purity of 92.5%, of which 25-40% was the “epi” or “cis” isomer of MDHJ, shown as Formula (3) below:

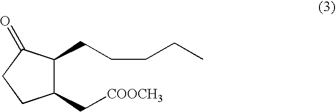

Unless otherwise noted, percentages, for example, percentages of additional or inert formulation ingredients, are given as percentages by volume.

### Example 1

### Improved Vase Life of Field Grown Roses After Treatment with MDHJ

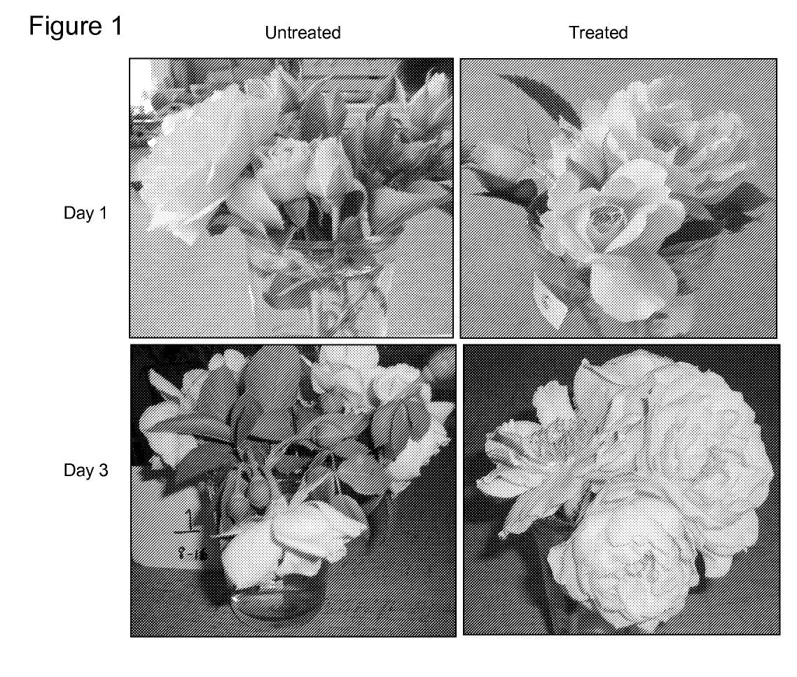

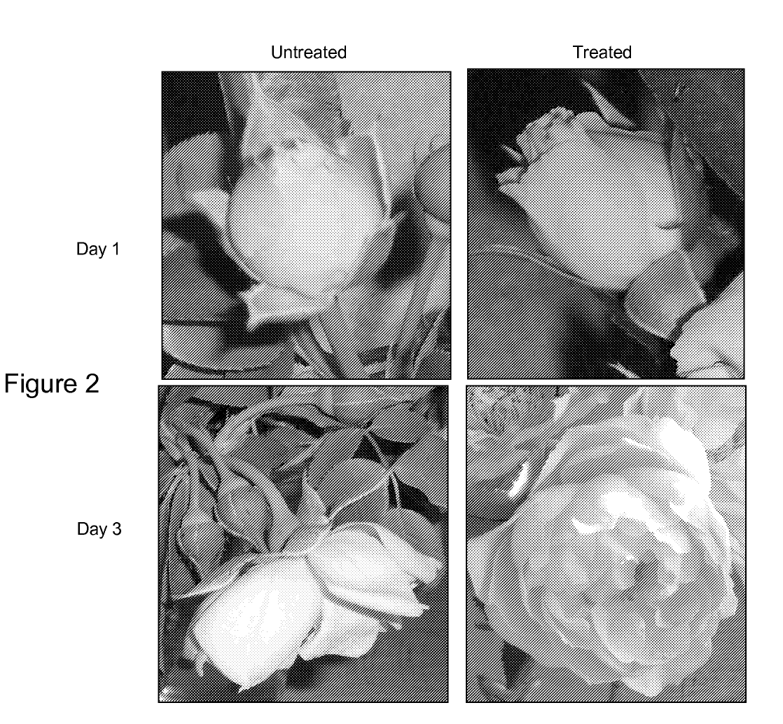

Foliage of field grown ‘Julia Child’ roses were sprayed to the point of drip 24 hours before harvesting with an aqueous solution comprising 5.0 mM MDHJ, 0.125% Triton® X-100, and 0.5% canola oil. On average, about 100 ml of the formulation was applied per plant, comprising an approximate volume of 130 μl (or 500 μM) MDHJ. Stems of flowers in the bud to early-bloom stage were cut in the field. Stems received a fresh cut while submerged under water before transferring to a water-filled vase. The bloom stage of 4-5 flowers was analyzed on day 1 and day 3, as compared with untreated roses that were otherwise similarly handled. The results are given in Table 2 below. FIG. 1 illustrates the full vases of flowers for the treated and untreated roses on day 1 and day 3, respectively. FIG. 2 illustrates individual roses on day 1 and day 3.

TABLE 2
Stages of Blooming in Treated and Untreated Rose.

Bloom Stage

| Treatment | Day 1 | Day 3 |
|---|---|---|
| Untreated | d | f |
| | b | f |
| | b | g |
| | b | g |
| | b | g |
| Treated | d | d |
| | c | d |
| | b | d |
| | b | d |

Key:
(a) Green Bud;
(b) Full Bud;
(c) Flower in early bloom stage;
(d) Flower in fully open stage;
(e) Flower in late bloom stage;
(f) Fully wilted flower;
(g) Flower drooped before fully opening.

The results of Example 1 demonstrate that treating plants with MDHJ before harvesting buds and early flowers improves the flowering characteristics by improving the ability of the buds to open fully into flowers. It also delays flower wilting and prevents stems from exhibiting bent neck.

### Example 2

### Prolonged Development of Flowers on Intact Rose Plants

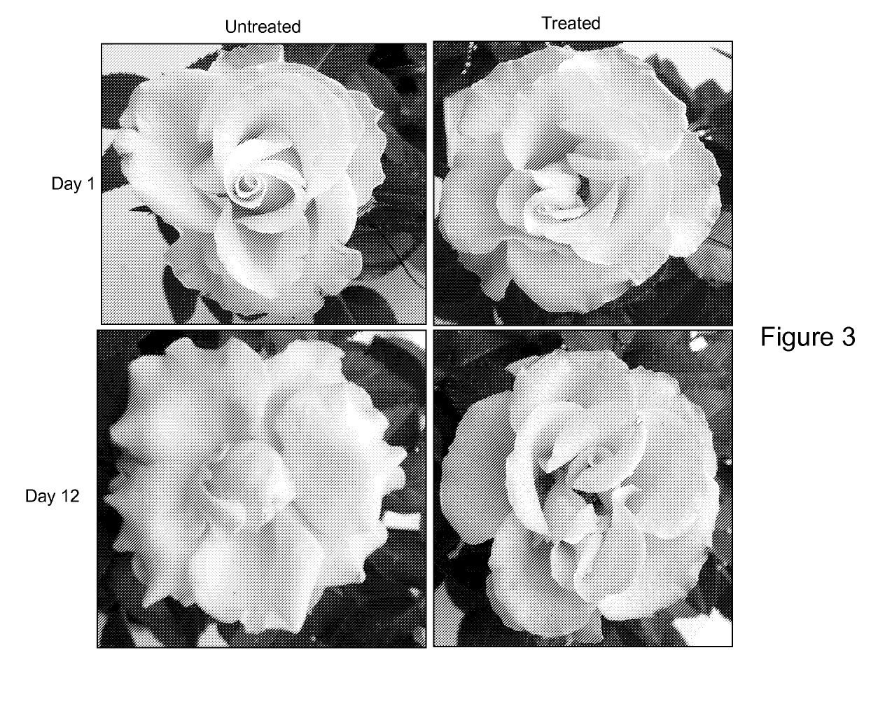

Parade Roses® with flowers in the bud to early bloom stage were treated to the point of drip on day 1 and day 2 with an aqueous solution comprising either (1) 0.125% Triton® X-100 and 0.5% canola oil as a control treatment; or (2) 1.5 mM MDHJ, 0.125% Triton® X-100, and 0.5% canola oil. There were four plants per treatment. Approximately 10 ml of each treatment was applied to each plant, comprising approximately 3.9 μL (15 μM MDHJ). Rate of flower development of individual flowers was monitored and compared between treatments. On day 1 and day 12, individual flowers were rated according to their stage of blooming. The results are given in Table 11 below. FIG. 3 is a photographic comparison of treated and untreated plants at day 1 and day 12.

TABLE 3
Effect of MDHJ on Flower Development in Intact Flowering Plants.

Bloom Stage

| Flower Number | Day 1 | Day 12 |
|---|---|---|
| Treatment 1 | | |
| 1 | 1 | 2 |
| 2 | 1 | 2 |
| 3 | 1 | 1 |
| 4 | 2 | 3 |
| 5 | 2 | 2 |
| 6 | 1 | 3 |
| 7 | 1 | 2 |
| Average | 1.3 | 2.1 |
| Treatment 2 | | |
| 1 | 2 | 2 |
| 2 | 1 | 1 |
| 3 | 2 | 2 |
| 4 | 1 | 1 |
| 5 | 1 | 1 |
| 6 | 2 | 2 |
| 7 | 1 | 1 |
| 8 | 1 | 1 |
| Average | 1.4 | 1.4 |

Key:
(1) Early bloom;
(2) Fully Open;
(3) Wilted. Treatment 1: 0.125% Triton ® X-100 + 0.5% canola oil; Treatment 2: 0.125% Triton ® X-100 + 0.5% canola oil + 1.5 mM MDHJ.

The results demonstrate that flowers from plants treated with a formulation containing MDHJ display prolonged flower development. Furthermore, the results indicate the effect is due to MDHJ and not the other ingredients.

While the invention has been described with respect to certain embodiments and examples, the description is intended to be illustrative, rather than limiting. Modifications and changes may be made within the scope of the invention, which is defined by the appended claims.

What is claimed is:

1. A method for improving flowering characteristics in a plant, comprising treating the plant with an effective amount of methyl dihydrojasmonate.

2. The method of claim 1, further comprising harvesting one or more flowers from the plant after treating the plant with the effective amount of methyl dihydrojasmonate.

3. The method of claim 1, wherein the effective amount of methyl dihydrojasmonate is in a form selected from the group consisting of emulsion, suspension, powder, hydrate, solution, granules, paste, aerosol, and volatile formulation.

4. The method of claim 3, wherein the methyl dihydrojasmonate is in solution with a compatible solvent.

5. The method of claim 4, wherein the compatible solvent comprises water.

6. The method of claim 5, wherein the effective amount of methyl dihydrojasmonate comprises an aqueous solution of methyl dihydrojasmonate with a concentration from about 0.15 mM to about 5 mM.

7. The method of claim 6, wherein the effective amount of methyl dihydrojasmonate comprises an aqueous solution of methyl dihydrojasmonate with a concentration of about 1.5 mM.

8. The method of claim 6, wherein the effective amount of methyl dihydrojasmonate comprises an aqueous solution of methyl dihydrojasmonate with a concentration of about 5 mM.

9. The method of claim 5, wherein the solution further comprises one or more additives selected from the group consisting of wetting agents, adjuvants, emulsifiers, dispersants, spreaders, surfactants, anchorage, and disintegrants.

10. The method of claim 9, wherein the solution further comprises a surfactant and an oil.

11. The method of claim 10, wherein the surfactant comprises Triton® X-100 and the oil comprises canola (rapeseed) oil.

12. The method of claim 1, wherein the flowering characteristics comprise one or more characteristics selected from the group consisting of duration that flowers last, degree to which buds open into full flower, and presence or absence of bent neck.

13. The method of claim 1, wherein the plant comprises a rose plant.

14. The method of claim 1, wherein the effective amount of MDHJ is in solid form with an amount of MDHJ in the range of about 0.008% to about 0.8% by weight.

15. A cut flower produced by a process comprising:
prior to cutting the flower from a plant, treating the plant with an effective amount of methyl dihydrojasmonate.

16. The cut flower of claim 15, wherein the effective amount of methyl dihydrojasmonate is in a form selected from the group consisting of emulsion, suspension, powder, hydrate, solution, granules, paste, aerosol, and volatile formulation.

17. The cut flower of claim 16, wherein the effective amount of methyl dihydrojasmonate comprises an aqueous solution of methyl dihydrojasmonate with a concentration from about 1.5 mM to about 5 mM.

18. The cut flower of claim 17, wherein the solution further comprises one or more additives selected from the group consisting of wetting agents, adjuvants, emulsifiers, dispersants, spreaders, surfactants, anchorage, and disintegrants.

19. The cut flower of claim 18, wherein the solution further comprises a surfactant and an oil.

20. The cut flower of claim 19, wherein the surfactant comprises Triton® X-100 and the oil comprises canola (rapeseed) oil.

21. The cut flower of claim 15, wherein the plant is treated with the effective amount of MDHJ about 24 hours prior to harvesting the cut flower.

22. The cut flower of claim 15, wherein the cut flower comprises a rose and the plant comprises a rose plant.

23. A method of improving vase life of cut roses, comprising:
prior to harvesting roses from a rose plant, treating the rose plant with an effective amount of methyl dihydrojasmonate; and
harvesting roses from the rose plant at a defined time after treating the plant.
